(19) United States
(12) Patent Application Publication
(10) Pub. No.: US 2015/0164413 A1
(43) Pub. Date: Jun. 18, 2015

(54) METHOD OF CREATING ANESTHETIC CONSCIOUSNESS INDEX WITH ARTIFICIAL NEURAL NETWORK

(71) Applicant: NATIONAL CHUNG SHAN INSTITUTE OF SCIENCE AND TECHNOLOGY, Longtan Township (TW)

(72) Inventors: SHANG-JU WU, LONGTAN TOWNSHIP (TW); NIEN-TZU CHEN, LONGTAN TOWNSHIP (TW); KUO-KUANG JEN, LONGTAN TOWNSHIP (TW); JIANN-SHING SHIEH, ZHONGLI CITY (TW); SHOU-ZEN FAN, TAIPEI CITY (TW)

(21) Appl. No.: 14/566,926

(22) Filed: Dec. 11, 2014

(30) Foreign Application Priority Data
Dec. 13, 2013 (TW) 102146017

(51) Int. Cl. A61B 5/00; A61B 5/11; A61B 5/0476

(52) U.S. Cl. CPC A61B 5/4821; A61B 5/0476; A61B 5/11; A61B 5/7203; A61B 5/7264

(57) ABSTRACT

A method of creating an anesthetic consciousness index with an artificial neural network includes, obtaining physiological signals, including electroencephalographic signals and eye movement signals, from subjects during a physiological signal monitoring process; filtering noise out of the physiological signals by empirical mode decomposition (EMD); calculating sample entropy values of the noise-removed physiological signals; obtaining sample entropy value sets of the physiological signals; repeating the aforesaid steps to effectuate measurement, noise-filtering, and sample entropy value calculation of the subjects' physiological signals and thus obtain a sample entropy value set; and applying an artificial neural network in conducting regression analysis of the sample entropy value set and a set of levels of consciousness measured with a physiological signal monitor during the physiological signal monitoring process, thereby creating the anesthetic consciousness index model for evaluating the level of consciousness of an anesthetized patient during the physiological signal monitoring process.

### CROSS-REFERENCE TO RELATED APPLICATION

This non-provisional application claims priority under 35 U.S.C. §119(a) on Patent Application No(s). 102146017 filed in Taiwan, R.O.C. on Dec. 13, 2013, the entire contents of which are hereby incorporated by reference.

### FIELD OF TECHNOLOGY

The present invention relates to methods of creating a consciousness index, and more particularly, to a method of creating an anesthetic consciousness index with an artificial neural network (ANN).

### BACKGROUND

Absolutely risk-free surgery never occurs, so does anesthesia taking place in an operating room, where top priority is given to the medical safety of an anesthetized patient undergoing an anesthetic procedure. To this end, physiological signal monitors are indispensable in operating rooms.

Typical examples of conventional physiological signal monitors include bi-spectral index (BIS) VISTA monitors manufactured by Aspect Medical Systems, and auditory evoked potential monitors (AEP monitors) manufactured by Alaris. As its name suggests, a BIS VISTA monitor analyzes and assesses electroencephalographically-depicted consciousness level in accordance with a bi-spectral index. An AEP monitor generates a sound wave for use as a stimulus to a patient in measuring variations of the patient's electroencephalographic potential to evaluate the patient's response to sound and thus evaluate the patient's anesthetic depth.

The bi-spectral index used by BIS VISTA monitors is predisposed to signal distortion in the presence of a functioning electrosurgical unit. Due to their overly low induced potential, the audio signals used by AEP monitors are susceptible to interference, especially electromagnetic interference caused by electrical appliances, thereby bringing inconvenience and limitations to surgical teams and setting strict operating room environment requirements. In addition, as its name suggests, AEP monitors work by sending auditory stimuli to patients and thus is inapplicable to patients with a hearing impairment.

In an attempt to overcome the drawbacks of BIS index and AEP index, scientists put forth evaluating anesthetic depth by analyzing brain waves with sample entropy. However, entropy values calculated by analyzing brain waves with sample entropy are riddled with problems, including noise, a lack of complete regression analysis, and a failure to display to observers (surgeons and nurses) efficiently and conveniently any data obtained.

### SUMMARY

It is an objective of the present invention to provide a complete analysis method which surpasses its conventional counterparts in determining a patient's anesthetic depth with a non-linear analysis technique like sample entropy, evaluating the patient's level of consciousness with a physiological signal monitor, and eventually conducting regression analysis to create an anesthetic consciousness index model conducive to determining the patient's anesthetic depth by anesthesiologists.

The present invention is based on non-linear analysis techniques, such as an index calculated by conventional sample entropy, and empirical mode decomposition (EMD), in comparison with the level of consciousness measured with a physiological signal monitor, and calculated with an artificial neural network (ANN), and is adapted to create an anesthetic consciousness index model, so as to enable surgeons and nurses to determine patients' level of consciousness precisely.

EMD was put forth by Chinese American N. E. Huang and the others in 1998 and is suitable for use in analyzing a non-linear, non-steady signal series, characterized by a high signal-to-noise ratio. The key to the method of the present invention lies in EMD whereby a complicated signal is decomposed into multiple intrinsic mode functions (IMF) each comprising local feature signals derived from the original signal and defined by different time dimensions. The process of extracting possible intrinsic mode functions from a signal is known as a sifting process. Two criteria must be met in order to extract possible intrinsic mode functions in the sifting process, otherwise it will be necessary for the possible intrinsic mode functions to undergo sifting again until the two criteria are met. The two criteria are: (a) in the whole time series, the difference between the total number of local extreme values and the total number of zero-crossings must not be larger than 1; and (b) at any point in time, an average envelope must approximate to zero.

If the aforesaid two criteria are met, the extracted signal is known as an intrinsic mode function (IMF) and marked as C1, whereas the signal obtained by subtracting C1 from the original signal is known as a residual signal. The residual signal serves as an input for use in obtaining the next intrinsic mode function by decomposition. The aforesaid process is repeated in order to gradually obtain different intrinsic mode functions by decomposition until the residual signal turns into a monotonic function.

EMD is conducive to stabilization of a non-stable data. As compared to short-time Fourier transform (STFT) and wavelet packet decomposition, EMD is intuitive, direct, and self-adaptive, not only because a base function is obtained by decomposing the data per se, but also because the decomposition takes place in accordance with local characteristics of signal series time dimensions and thus is self-adaptive.

Entropy, a concept in physics, is a measure of the disorder or randomness in a closed system. From a perspective of information, entropy describes how irregular, intricate, and unpredictable a signal is. Entropy is calculable in terms of time, frequency, or both.

Sample entropy, which enables signals to be analyzed in terms of time, is similar to approximation entropy which is also applicable to time. Sample entropy features self-exclusion and aims to improve on approximation entropy. Sample entropy involves calculating the probability of generating a signal from a non-linear system, so as to define the regularity and complexity of a system in a quantified manner. The higher the sample entropy, the lower the self-similarity of a series, the higher the probability of generating a new signal, the more complicated the series. Conversely, the lower the sample entropy, the higher the self-similarity of a series, the lower the probability of generating a new signal, the simpler the series.

A sample entropy value has a numerical range of 0 to 3 approximately. To allow physicians and the other medical professionals to evaluate a patient's anesthetic depth in a conventional way, it is feasible to apply an artificial neural network (ANN) in conducting regression analysis of a set of levels of consciousness and anesthetic consciousness levels measured with a physiological signal monitor such that the sample entropy value has a numerical range of 0 to 100.

The artificial neural network is a parallel calculation model and operates in a manner similar to human beings' neurological operating mechanism; hence, the artificial neural network is also known as a parallel distributed processing model or a connectionist model. The artificial neural network requires undergoing learning iteratively and correcting errors repeatedly with a view to attaining an optimal output and drawing the best conclusion as effectively as the human brain does.

The typical learning process carried out with the artificial neural network comes in three patterns, namely supervised learning, unsupervised learning, and reinforced learning.

The training process of supervised learning involves generating a new weight in accordance with rules of correlation between an input value and an output value, such as back propagation network (BPN), learning vector quantization (LUQ), and counter propagation network (CPN).

The training process of reinforced learning involves judging the degree of importance of output values and then generating a new weight in accordance with the difference in importance. Although reinforced learning shares the same target of comparison with supervised learning, reinforced learning fails to cast any light on the actual output. As a result, reinforced learning usually requires full access to the degree of importance in order to effectuate feedback by correcting a weight better.

The training process of unsupervised learning requires the input value only and entails generating new weights by learning the rules of internal clustering of data. Examples of unsupervised learning include self-organization map (SOM) and adaptive resonance theory (ART).

The artificial neural network of the present invention comprises an input layer, a hidden layer, and an output layer, in order to fetch a training template and a target output value from an issue domain by supervised learning and back propagation network (BPN), input the training template to a network, and adjust the connection weight and bias of the network iteratively, so as to approximate to a physician's diagnosis.

### BRIEF DESCRIPTION

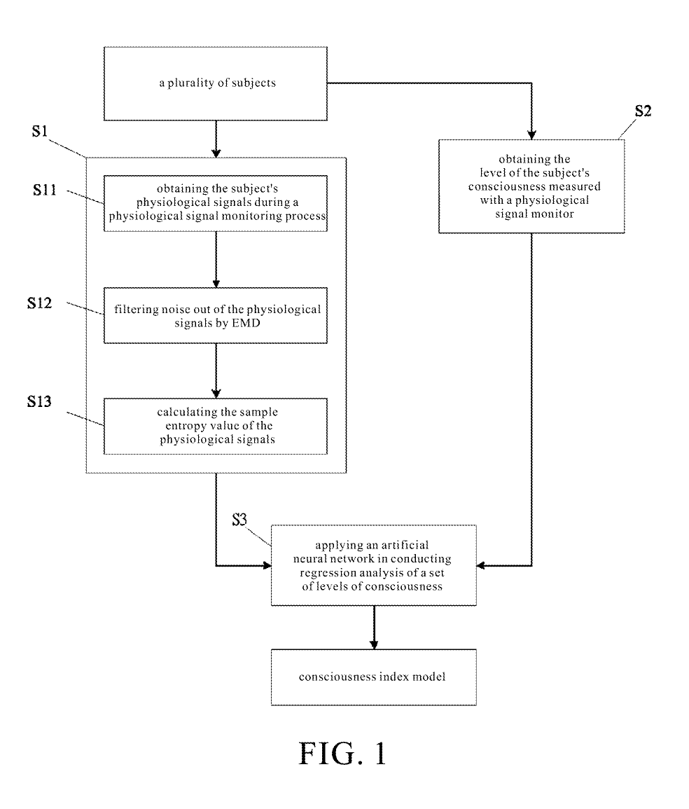

FIG. 1 is a flow chart of a method of creating an anesthetic consciousness index model with an artificial neural network according to an embodiment of the present invention.

### DETAILED DESCRIPTION

Referring to FIG. 1, there is shown a flow chart of a method of creating an anesthetic consciousness index model with an artificial neural network according to an embodiment of the present invention. The process flow of the method is described below, as shown in FIG. 1.

Step S11: capture a plurality (N) of physiological signals from a subject during a physiological signal monitoring process performed on the subject. The physiological signals are each an electroencephalographic signal or an eye movement signal. N correlates with the duration of the physiological signal monitoring process and the sampling rate of capturing the physiological signals from the subject.

Step S12: filter, by empirical mode decomposition (EMD), noise out of the N physiological signals captured during the physiological signal monitoring process, so as to obtain noise-removed physiological signals.

Step S13: perform sample entropy value calculation on the noise-removed physiological signals. Assuming that the physiological signal monitoring process yields N data, it is feasible to predetermine the number of data to be compared among the 1^{st} to n^{th} noise-removed physiological signals, wherein n correlates with the sampling rate. Given a sampling rate of 125 Hz, n ranges from 10^{m} to 30^{m}, where m denotes the predetermined number of data to be compared among SampEn(N, m, r). Given m=2, then n ranges from 100 to 900 and is preferably the average 500, and thus, given the sampling rate of 125 Hz, the predetermined number of data to be compared amounts to the number of the physiological signals captured in four seconds. Step S13 further involves calculating a sample entropy value, calculating the 2^{nd} to n+1^{th} physiological signals by scrolling a window, calculating a sample entropy value, and so on to thereby calculate N−n+1 sample entropy values (indicative of the patient's consciousness index during the physiological signal monitoring process.) In this embodiment, SampEn(N, m, r) expresses a sample entropy, where m denotes the predetermined number of data to be compared, r denotes the predetermined tolerance range coefficient, and N denotes data cycle length. The sample entropy is calculated as follows:

The original data is expressed by x(1), x(2), . . . , x(N).

(1) The original data brings about m-dimensional vectors, i.e., uₘ(1) to uₘ(N−m), where uₘ(i)=[x(i), x(i+1), . . . , x(i+m−1)], i=1˜N−m+1

(2) The distance between uₘ(i) and uₘ(j) is defined as d[uₘ(i), uₘ(j)] and equals the largest difference between the equivalent elements of uₘ,(i) and uₘ(j).

d[uₘ(i), uₘ,(j)]=max{|x(i+k)−x(j+k)|: 0≦k≦m−1}

(3) Given threshold R (R=r*SD, SD denotes the standard deviation of the original series), with 1≦i≦N−m, calculate the number of d[uₘ,(i), uₘ,(j)] smaller than R and divide it by N−m to obtain B^{m}(r). Its equation is as follows:

$${B}^{m} ( r ) = {( N - m )}^{- 1} {\sum }_{i = 1}^{N - m} {B}_{i}^{m} ( r )$$

(4) Increase the dimension by 1, and repeat steps (1)˜(3) to obtain A^{m}(r). Its equation is as follows:

$${A}^{m} ( r ) = {( N - m - 1 )}^{- 1} {\sum }_{i = 1}^{N - m} {A}_{i}^{m} ( r )$$

(5) B^{m}(r) and A^{m}(r) denote the probability of similarity between two series in m dimensions and m+1 dimensions, respectively, wherein, when N is finite, the sample entropy is calculated with the equation below.

$$SampEn ( N , m , r ) = - log \frac{{A}^{m} ( r )}{{B}^{m} ( r )}$$

The physiological signals of each subject during the physiological signal monitoring process are captured, and then step S11 to step S13 are repeated, so as to obtain consciousness indexes expressed by a plurality of sample entropy values, respectively.

Step S3: combine the plurality of consciousness indexes obtained in step S1 and the plurality of consciousness indexes obtained in step S2 using a physiological signal monitor, and then perform regression analysis of the combined consciousness indexes with an artificial neural network to create a consciousness index model for determining patients' level of consciousness precisely.

In this embodiment, the EMD for use in step S12 entails decomposing the original signal into a plurality of intrinsic mode functions (IMF) each comprising local feature signals derived from the original signal and defined by different time dimensions. The intrinsic mode functions are identified in accordance with two boundary conditions: (a) in the whole time series, the difference between the total number of local extreme values and the total number of zero-crossings must not be larger than 1, that is, each extreme value is immediately followed by a zero-crossing; and (b) at any point in time, an average envelope must approximate to zero. According to the aforesaid two boundary conditions, original data x(t) is converted into intrinsic mode functions with EMD by creating upper and lower envelopes, that is, identifying the local maxima and local minima of original data x(t), connecting the local maxima of original signal x(t) to form an upper envelope, and connecting the local minima of original signal x(t) to form a lower envelope. Then, averages are calculated, wherein the curve of the averages of the original data x(t) expresses the average of the upper and lower envelopes and is denoted by m₁(t).

Afterward, the sifting process is carried out repeatedly. The first instance of sifting entails subtracting the average m₁(t) from original data x(t) to obtain the first component signal h₁(t). Then, each following instance of sifting entails repeating the first instance of sifting to thereby allow the average mₖ(t) to approximate to zero, wherein the number of extreme values gradually approximates to the number of zero-crossings. The sifting process is expressed by equations as follows:

x(t)−m₁(t)=h₁(t)

h₁(t)−m₂(t)=h₂(t)

hₖ₋₁(t)−mₖ(t)=hₖ(t)

→hₖ(t)=c₁(t)

The sifting process further comprises the step of determining whether the component signal resulting from the sifting process satisfies the aforesaid boundary conditions, as described below. When the sifting process is underway, it is necessary to compare each component signal hₖ(t) and each of the two aforesaid boundary conditions. If the component signal meets the two boundary conditions, the component signal will be regarded as an intrinsic mode function expressed by hₖ(t)=c₁(t). At this point in time, the sifting process is done.

After an intrinsic mode function has been obtained, it is necessary to separate it from original data x(t) to thereby obtain a residual r₁(t). The residual r₁(t) is indicative of a signal with a long cycle and a low frequency. The residual r₁(t) is regarded as a new original data. The aforesaid separation step is repeated several times to thereby obtain several intrinsic mode functions which decrease successively in frequency. The separation step is expressed by equations as follows:

$$x ( t ) - {c}_{1} ( t ) = {r}_{1} ( t )$$

$${r}_{1} ( t ) - {c}_{2} ( t ) = {r}_{2} ( t )$$

$$…$$

$${r}_{n - 1} ( t ) - {c}_{n} ( t ) = {r}_{n} ( t ) \\ -> x ( t ) - {\sum }_{k = 1}^{n} {c}_{k} ( t ) = {r}_{n} ( t )$$

What is claimed is:

1. A method of creating an anesthetic consciousness index model with an artificial neural network, the method comprising the steps of:
(a) obtaining a plurality of physiological signals from a subject during a physiological signal monitoring process;
(b) filtering noise out of the plurality of physiological signals by empirical mode decomposition (EMD);
(c) calculating a plurality of sample entropy values of the plurality of noise-removed physiological signals;
(d) generating a sample entropy value set of the physiological signals from a plurality of subjects during a physiological signal monitoring process by repeating steps (a)˜(c); and
(e) performing, with an artificial neural network algorithm, regression analysis of the subjects' sample entropy value set and the subjects' anesthetic consciousness levels measured with a physiological signal monitor, so as to obtain an anesthetic consciousness index model.

2. The method of claim 1, wherein the physiological signals are each one of an electroencephalographic signal and an eye movement signal.

3. A method of creating an anesthetic consciousness index model with an artificial neural network, the method comprising the steps of:
(a) obtaining a plurality of physiological signals from a subject during a physiological signal monitoring process;
(b) filtering noise out of the plurality of physiological signals by empirical mode decomposition (EMD);
(c) calculating a sample entropy value of 1^{st} to n^{th} said noise-removed physiological signals, wherein n correlates with a sampling rate;
(d) calculating a next sample entropy value of 2^{nd} to n+1^{th} physiological signals by repeating step (c);
(e) repeating step (c) until all the physiological signals of the subjects during a physiological signal monitoring process have been processed;
(f) repeating steps (a)˜(e) to process multiple subjects' physiological signals during a physiological signal monitoring process and thus generate a sample entropy value set; and
(g) performing, with an artificial neural network algorithm, regression analysis of the subjects' sample entropy value sets and the subjects' anesthetic consciousness levels measured with a physiological signal monitor, so as to obtain an anesthetic consciousness index model.

4. The method of claim 3, wherein the physiological signals are each one of an electroencephalographic signal and an eye movement signal.

5. A method of creating an anesthetic consciousness index model with an artificial neural network, the method comprising the steps of:
(a) obtaining a plurality of physiological signals from a plurality of subjects during a physiological signal monitoring process, respectively;
(b) filtering noise out of the physiological signals by empirical mode decomposition (EMD);
(c) generating a sample entropy value set of each subject's physiological signals by performing the following steps;
(i) calculating a sample entropy value of 1^{st} to n^{th} said noise-removed physiological signals, wherein n correlates with a sampling rate;
(ii) calculating a next sample entropy value of 2^{nd} to n+1^{th} physiological signals by repeating step (i); and
(iii) repeating step (i) until all the physiological signals of the subjects during a physiological signal monitoring process have been processed; and
(d) performing, with an artificial neural network algorithm, regression analysis of the subjects' sample entropy value sets and the subjects' anesthetic consciousness levels measured with a physiological signal monitor, so as to obtain an anesthetic consciousness index model.

6. The method of claim 5, wherein the physiological signals are each one of an electroencephalographic signal and an eye movement signal.
